(19) United States
(12) Patent Application Publication
(10) Pub. No.: US 2002/0044972 A1
(43) Pub. Date: Apr. 18, 2002

(54) Polymer compositions for polynucleotide delivery

(75) Inventors: Stanley Stewart Davis, Nottingham (GB); Lisbeth Illum, Nottingham (GB); Burhan Daudali, Nottingham (GB)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: West Pharmaceutical Services Drug Delivery & Clinical Research Centre, Ltd.

(21) Appl. No.: 09/944,291

(22) Filed: Aug. 31, 2001

Related U.S. Application Data
(63) Continuation of application No. PCT/GB00/00665, filed on Feb. 24, 2000.

(30) Foreign Application Priority Data
Mar. 2, 1999 (GB) 9904627.8

(51) Int. Cl. A61K 48/00; A61K 9/14; C08G 59/14

(52) U.S. Cl. 424/486; 514/44; 525/54.3

(57) ABSTRACT

A composition is provided including: (a) a nucleic acid or an oligonucleotide; and (b) a block copolymer containing a hydrophilic block that carries functional groups that provide the block with a positive charge. These compositions may be used to deliver a nucleic acid or an oligonucleotide to a cell.

### CROSS-REFERENCE TO RELATED APPLICATION

This application is a continuation of International Application No. PCT/GB00/00665, filed Feb. 24, 2000, the disclosure of which is incorporated herein by reference, which was published in the English language on Sep. 8, 2000 under International Publication No. WO 00/51645.

### BACKGROUND OF THE INVENTION

The present invention relates generally to the delivery of polynucleotides in the form of oligonucleotides (antisense agents) and nucleic acids (DNA). More specifically, the present invention relates to a composition comprising a nucleic acid or oligonucleotide and a block copolymer containing a hydrophilic block that carries functional groups that provide the block with a positive charge.

The binding of oligonucleotides to specific nucleic acid sequences may inhibit the interaction of RNA with proteins, other nucleic acids or other factors that are essential for metabolism in a cell and thereby provide a clinically relevant effect, for example oligonucleotides (antisense agents) can be useful in cancer treatment, as antivirals, and in the modification of the inflammatory processes. Gene therapy offers a means of treating a variety of diseases and a means for vaccinations.

For antisense and gene therapy to be successful it is essential that the polynucleotide be delivered into a target cell. This can be achieved using a delivery system, more often known as a vector. Such vectors can be in the form of a virus particle (carrying DNA) or a non-viral vector.

An essential attribute of a non-viral vector is an ability to compact an oligonucleotide or plasmid DNA into a small particle, preferably carrying a positive charge. The prior art describes different approaches, which are largely based on cationic lipids and cationic polymers. For example, see Antisense Research and Application., Ed. Cooke ST, Springer, Berlin (1998); J. Drug Target., Special issue on Drug Delivery and Targeting of Oligonucleotide Based Therapeutics, Vol.5. (1998); Artificial Self Assembly Systems for Gene Therapy, Felgner et al. Editors, ACS Conference Services, ACS Washington (1996); Delivery Strategies for Antisense Oligonucleotide Therapeutics, Editor Akhtar S., CRC Press, Boca Raton (1995); Self-Assembling Complexes for Gene Delivery, Kabanov et al. Editors, Wiley, London (1998).

One of the earliest cationic polymers to be employed for polynucleotide delivery was polylysine. This polymer can be obtained in different molecular weights. By mixing polylysine with oligonucleotides or plasmid DNA it is possible to produce small particles in the size range 10 to 1000 nm. These particles are termed “nanoparticles”. Such nanoparticles can be used to transfect cells in vitro as well as in vivo. However, polylysine is toxic and as a consequence, others have employed alternative cationic materials, such as polyamidoamines, polyglucosamine (Chitosan) and polyethyleneimines. The principle is the same as for polylysine in that the cationic polymer interacts with the anionic polynucleotide to produce an insoluble complex that comes out of solution as a nanoparticle.

The size and surface charge on the nanoparticle can be controlled by various factors, which include the concentration of the interacting species, the pH and ionic strength of the interaction medium, the rate of addition of one component to the other, the molecular weight and structure of the cationic polymer.

The formed nanoparticles must be stable in a biological environment (especially in the presence of serum) and they must produce efficient transfection of target cells. However, in some cases, nanoparticles can be taken up by target cells, but transfection is inefficient. This has been associated with the fate of the nanoparticle in the cell and in particular its fate in the endosomal compartment. It is necessary that the polynucleotide can leave the endosome after uptake and transverse the cytoplasm and nuclear membrane to reach the cell nucleus. In order to effect release of the polynucleotide from the endosome, lytic peptides or the lysosomotrophic agent chloroquine can be employed. While these approaches are possible in vitro or ex vivo, they have little utility in vivo.

In the field of gene therapy, WO 96/15778 describes how unmodified block copolymers of the poloxamer or poloxamine type (i.e. polyalkylene block copolymers composed of polyoxyethylene and polyoxypropylene) can be used to provide transfection of cells. A plasmid is first complexed with a polycation. The amounts of the plasmid and polycation are calculated to provide a ratio of polycation basic groups to plasmid phosphate groups of about 1 to 10. A poloxamer is then added, the ratio of the poloxamer to DNA being about 1 to 10⁴.

WO 96/15778 also describes a polynucleotide complex between a copolymer comprising a polyether block and a polycation block, such as polyoxyethylene-poly-L-lysine.

The preparation and properties of polyoxyalkylene block co-polymers have been described by Nace, Non-Ionic Surfactants, Polyoxyalkylene Block Co-Polymers, Dekker, New York (1996). The poloxamers (CAS-93003-11-6) (Pluronic™) comprise two polyoxyethylene blocks and a polyoxypropylene blocks (see for example, Schmolka in Polymers for Controlled Drug Delivery, p. 189-214, Tarcha, P. editor, CRC Press, Boca Raton, Fla. (1991). The poloxamers, which comprise a star shaped molecule with four ethylene oxide blocks, are attached to polyoxypropylene blocks through a central ethylene diamine function.

Erbacher et al., Bioconj. Chem., 6:401 (1995) describes glycosylated polylysine-DNA complexes. A reduction of the positive charges on polylysine by partial gluconylation has been reported to increase the transfection efficiency of polylysine DNA complexes (Biochem. Biophys. Acta, 1324:27 (1997)).

A major problem with the in vivo delivery of polynucleotides is that after administration of compacted nanoparticles, the vector may not deliver the polynucleotide to the intended site but instead the material can be captured by the defense system of the body; the reticuloendothelial system. For example, a DNA-polymer nanoparticle, injected intravenously into the blood stream, will be largely sequestered by the macrophages present in the liver (Kupffer cells) and to a lesser extent, by the spleen. It is known that the capture of nanoparticles can be minimised by the attachment of hydrophilic moieties to the surface of particles as described in U.S. Pat. No. 4,904,479 and more recently as the so called ‘stealth liposome concept’. U.S. Pat. No. 4,904,479 describes the use of polyethylene glycol (PEG) to prevent such capture.

WO 97/25067 describes polyamidoamine-PEG polymers and describes how PEG modified cationic polymers can be used to compact DNA to produce nanoparticles that carry PEG groups on their surface.

Wolfert et al., Hum. Gene Ther., 7:2123 (1996) and Katayase and Kawabata, J. Pharm. Sci., 87:160 (1996) have synthesized simple A-B type copolymers of PEG and poly-L-lysine (PLL). These polymers were interacted with DNA.

It is believed that PEG modified polynucleotide nanoparticles will have extended circulation times in the blood if they are sufficiently stable. By the term sufficiently stable we mean that the oligonucleotide or DNA, and cationic polymer have a sufficiently strong interaction to prevent their disruption by plasma components for more than 5 minutes, preferably for more than 10 minutes and most preferably for more than 30 minutes. The PEG groups on the surface of the nanoparticles may also be useful in reducing the degradation of the DNA by serum nucleases.

Neal et al., J. Pharm. Sci., 87:1242 (1998) describes aminated block copolymers as a means for following the biodistribution of polymeric coating materials.

Wu et al., J. Biol. Chem., 262:4429 (1987) describes polylysine attached to asialoglycoprotein, which acts as a target in gene therapy.

There is a need for a cationic polymer, which has low toxicity and which is able to compact plasmid antisense oligonucleotides and DNA into a nanoparticle and provide cell transfection without the need for agents such as chloroquine.

A person of ordinary skill in the art will appreciate that the considerations that can be applied to the delivery of antisense oligonucleotides to the nucleus of a cell can also be applied to DNA.

### BRIEF SUMMARY OF THE INVENTION

The present applicant has developed a novel non-viral vector in the form of a composition comprising a nucleic acid or an oligonucleotide and a block copolymer containing a hydrophilic block that carries functional groups that provide the block with a positive charge. The composition may be used for the delivery of a nucleic acid or oligonucleotide to a cell.

According to the present invention, there is provided a composition comprising a nucleic acid or oligonucleotide and a block copolymer containing a hydrophilic block that carries functional groups that provide the block with a positive charge.

The net positive charge on the modified block copolymer enables it to interact with an oligonucleotide or DNA to form nanoparticles.

The present invention also provides a composition comprising a nucleic acid or oligonucleotide and a block copolymer containing a hydrophilic block, wherein the hydrophilic block has been aminated.

### DETAILED DESCRIPTION OF THE INVENTION

In a preferred embodiment of the present invention, there is provided a composition adapted for the delivery of a nucleic acid or oligonucleotide to a cell comprising a nucleic acid or oligonucleotide and a block copolymer containing a hydrophilic block that carries functional groups that provide the block with a positive charge, wherein the block copolymer also carries a targeting moiety.

The targeting moiety is typically attached to the modified block copolymers via at least some of the aminated hydrophilic groups.

The targeting moiety provides the ability to target specific cells. Instead of the nanoparticles circulating in the blood, they are targeted to a specific cell type. For example, in gene therapy it would be advantageous to target DNA to the hepatocytes of the liver. In order to achieve this targeting the particles need to be small (i.e., 500 nm or less in diameter) in order to escape from the liver sinusoids through to the space of Disse and to be in contact with the target cells.

Hepatocytes carry receptors for sugars such as galactose. Therefore, to aid the uptake of DNA by the hepatocytes of the liver the nanoparticles can be provided with a sugar moiety as a targeting moiety. A preferred targeting moiety is galactose.

The sugar can be attached to at least some of the aminated hydrophilic groups on the aminated block copolymers by a process known as glycosylation.

The process of glycosylation should leave the block polymer with a net positive charge to allow interaction with an oligonucleotide or DNA.

Preferably, no more than 95% of the amino groups should be glycosylated with a sugar moiety. More preferably, no more than 80% of the amino groups should be glycosylated with a sugar moiety, and it is especially preferred that no more than 50% of the amino groups should be glycosylated with a sugar moiety.

The attachment of sugars to the modified block copolymers can result in an improved uptake of plasmid DNA into target cells in the form of cultured hepatocytes. A preferred targeting moiety for hepatocyte targeting in the liver is galactose. A preferred targeting moiety for targeting to endothelial cells is fucose.

The person of ordinary skill in the art will appreciate that a range of targeting moieties can be chosen, such as monoclonal antibodies, or fragments thereof. Lectins and carbohydrates such as selectins can also be used depending on nature of the target cells.

The use of targeting moieties can result in an improved uptake of plasmid DNA into target cells such as cultured hepatocytes.

In another embodiment of the present invention, there is provided a composition adapted for the delivery of a nucleic acid or oligonucleotide to a cell comprising a nucleic acid or oligonucleotide and a block copolymer containing a hydrophilic block that carries functional groups that provide the block with a positive charge and a hydrophobic block.

When the block copolymer contains a hydrophilic block it may optionally also carry a targeting moiety. In this embodiment, the targeting moiety is attached to the copolymer via. cationic functional groups carried by the hydrophilic group.

Block copolymers that are suitable for use in the present invention include copolymers having ABA structures, where A refers to a hydrophilic block and B to a second, preferably hydrophobic, block. The polymers can alternatively have AB structures, wherein A is a hydrophilic block and B block is, for example, polylactide or polyoxypropylene.

Hydrophilic blocks suitable for use in the present invention include polyoxyethylene and dextran. A preferred hydrophilic block is polyethylene glycol.

Hydrophobic blocks that are suitable for use in the present invention include polyoxypropylene, polyoxybutylene and polylactic acid. A preferred hydrophobic block is polyoxypropylene.

Block copolymers that are especially preferred for use in the present invention include polyalkylene block copolymers that are composed of polyoxyethylene and polyoxypropylene blocks (known as poloxamines and poloxamers). Polyoxyethylene-lactic acid block copolymers are also preferred.

The nature and properties of the block copolymers which are suitable for use in the present invention are not particularly limited. Suitable block copolymers are available with a wide range of molecular structures and properties because the sizes of the polyoxyethylene and polyoxypropylene moieties can be varied and a wide variety of oxide type, oxide ratio and molecular weight are available.

Block copolymers that are preferred for use in the present invention include copolymers that are readily soluble in water and which have an ethylene oxide content of greater than 50%. Block copolymers with an ethylene oxide content of 80% are especially preferred.

The molecular weight of the polyoxypropylene block can be from 1000 to 6000 Daltons, in the poloxamer series and from 750 to 7000 Daltons in the poloxamine series.

Block copolymers that are especially suitable for use in the present invention include poloxamers 188, 288, 338, 407 and poloxamine 908.

Further details of suitable polyoxamers and poloxamines can be found in Surfactant Systems, p. 356-361, Eds. Attwood and Florence, Chapman and Hall, London (1983); The Condensed Encyclopaedia of Surfactants, Ed. Ash and Ash, Edward Arnold, London (1989); and Non-Ionic Surfactants, Ed. Nace, Dekker, New York (1996).

The hydrophilic block is modified so that it carries a positive charge. Preferably, the functional groups carried by the hydrophilic block are amine functional groups. Aminated poloxamers and poloxamines are especially preferred copolymers for use in the present invention. These aminated copolymers can be obtained by a process of substitution of the terminal hydroxyl group by an amino group. This process is known as “amination”.

The interaction of the aminated (and optionally glycosylated) polymer with a polynucleotide can be controlled by the choice of the block copolymers (that are available in different molecular weights and different ratios of polyoxyethylene to polyoxypropylene).

The mean diameter or particle size (as measured by light scattering or photon correlation spectroscopy or turbidimetric evaluation) of the nanoparticles formed between polynucleotides and the modified block copolymers is from 10 nm to 1000 nm. Preferably the mean diameter is 500 nm or less. A mean diameter of from 20 to 500 mn is preferred and a mean diameter of from 50 to 250 nm is especially preferred.

The nanoparticles can be formed by the admixture of solutions of the polynucleotide and modified block copolymer. Suitable solvents include water and buffer solutions. Typically the nanoparticles precipitate to provide a turbid suspension. The nanoparticles can be removed from the suspension using techniques standard in the art.

The amount of modified block copolymer present in the nanoparticles is generally greater than the amount of polynucleotide. The weight ratio of polynucleotide to block copolymer is typically from 1:5000 to 1:5. A preferred weight ratio of polynucleotide to block copolymer is from 1 to 100, and an especially preferred weight ratio is from 1 to 50.

The concentration of the polynucleotide used for the interaction can be from 0.1 mg/ml to 100 mg/ml. A preferred concentration of the polynucleotide is from 0.5 mg/ml to 10 mg/ml.

The concentration of the block copolymer can be from 1 mg/ml to 100 mg/m. A preferred concentration of the block copolymer is from 5 mg/ml to 50 mg/ml.

The charge on the resultant nanoparticle as measured by the technique of microelectrophoresis using, for example, the Malvern Zetasizer (laser doppler anenometry) can be from −20 mV to +100 mV at pH 7 at an ionic strength of 0.001 molar. A preferred charge on the nanoparticle is from 1 to 50 mV at the same conditions of pH and ionic strength.

The molecular weight of the block copolymer can be from 1 to 500 kd. A molecular weight of the block copolymer from 5 to 100 kd is preferred.

The present invention also provides a glycosylated block copolymer. The glycosylated block copolymer of the invention may comprise a hydrophilic block and a hydrophobic block. The sugar moieties are typically attached to the copolymer via cationic functional groups carried by the hydrophilic block. Preferably, the hydrophilic block is a polyoxyethylene block and the hydrophobic block is a polyoxypropylene block.

The present invention also provides a method for the delivery of a nucleic acid or an oligonucleotide to cells which comprises administering a composition of the invention.

Further, the present invention provides a method for targeting a nucleic acid or oligonucleotide to the liver using a glycosylated block copolymer.

The compositions and glycosylated block copolymers of the invention may be used in the manufacture of medicaments for the delivery of a nucleic acid or an oligonucleotide to a cell.

The compositions of the invention can be administered to a patient using techniques well known in the art. They may be administered by injection which may, for example, be intramuscular, intravenous, subcutaneous, intraarticular or intraperitoneal. The compositions may be administered to the dermal or epidermal layer of the skin by injection or needleless injector system. Alternatively, they may be administered to mucosa such as the nose, the gastrointestinal tract, the colon, the vagina and the rectum.

The compositions of the invention can be formulated in ways well known in the art.

The formulations may conveniently be presented in unit dosage form and may be prepared by any of the methods well known in the art of pharmacy. Such methods include the step of bringing the compositions into association with a suitable carrier, which constitutes one or more accessory ingredients. In general, the formulations are prepared by uniformly and intimately bringing the compositions into association with liquid carriers or finely divided solid carriers or both, and then, if necessary, shaping the product.

Formulations suitable for parenteral administration include, but are not limited, to aqueous sterile injection solutions which may contain anti-oxidants, buffers, bacteriostats and solutes which render the formation isotonic with the blood of the intended recipient; and aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that, in addition to the ingredients particularly mentioned above, the formulations of this invention may include other agents conventional in the art having regard to the type of formulation in questions.

The amount of the composition of the invention to be administered to a patient may be determined in relation to the amount of active agent to be administered and to the amount of active agent present in the composition of the invention and to the way in which the active agent becomes available in the patient following administration of the composition.

Suitably, the amount of the composition administered is from 1% to 1000% of the normal amount of the active agent administered to the patient when administered in a conventional way.

Preferably, the amount of active agent is from 10% to 500% of the normal amount of the active agent; more preferably from 20% to 80%.

For nasal administration, the vaccines can be administered as a fine suspension using a spray device or if in the form of a powder using a power device or nasal insufflator. Such devices are familiar to those skilled in the art.

The compositions of the invention may also be administered orally. Compositions for oral administration may be in any form known in the art, for example tablets, capsules, compressed or extruded pellets, suspensions or solutions.

For surface adsorbed antigens that are sensitive to the acid conditions in the stomach the delivery system can be protected by an enteric polymer familiar to those skilled in the art of formulation. The enteric polymer can be used to coat the dosage form.

Vaginal systems suitable for delivery include gels and vaginal suppositories. Rectally administrated vaccines can be given as enemas or incorporated into suppositories.

The present invention is now illustrated, but not limited, with reference to the following Examples. The block copolymer poloxamine 908 is used in the examples, but other block copolymers of the poloxamine series or poloxamer series could be employed.

### EXAMPLE 1

### Amination of Poloxamine

The method described by Neal et al., J. Pharm. Sci., 87:1242 (1998) was employed to modify the terminal hydroxyl groups of poloxamine 908 by an amino group.

Poloxamine 908 was obtained from BASF. A 20% w/v solution of the copolymer in CH₂Cl₂ was reacted with a two-fold excess of p-toluenesulphonyl chloride and pyridine at room temperature for 24 hours. The p-toluenesulphonate ester product was recovered by first washing with 3M HCl, followed by washing the organic layer with NaHCO₃. Rotary evaporation was used to obtain the co-polymer. In the second step, the p-toluenesulphonate ester product was reacted with 25% w/v NH₃ in H₂O for 6 hours at 120° C. in a pressurised reaction vessel, to produce the aminated copolymer. The reaction products were cooled to room temperature and extracted with CH₂Cl₂ to separate the ammonium toluenesulphonate salt from the aminated copolymer. The product was then washed with base (NaOH/H₂O) to produce the free amino product, which was recovered by solvent removal.

End group conversion was analysed by ¹H NMR analysis of the tosylated intermediates, using trichloroacetyl isocyanate (TAIC) labelled polymers. TAIC reacts with the terminal hydroxyl group to give a shift in NMR peak of the alpha-methylene protons adjacent to the hydroxyl groups. However, with the tosylated copolymers, no shift was detected, confirming complete end group conversions.

### EXAMPLE 2

### Synthesis of Galactosylated Poloxamine 908

The process of reductive amination was used to link lactose onto the aminated poloxamine 908, as this method preserves the cationic charge of the aminated poloxamines. Tetra amine poloxamine 908 (TA908), as produced in method described in Example 1, lactose (165 mg) and sodium cyanoborohydrate (112 mg) were dissolved in 10 ml of 0.2M phosphate buffer pH 9.2. The solution was heated to approximately 70° C. to completely dissolve the reactants. The mixture was then kept at 35 to 40° C. for 48 hours. The temperature was then raised to 60° C. for 24 hours, then to 95° C. for a brief period. The reaction products were cooled to room temperature and extracted with CH₂Cl₂ to separate the galactosylated poloxamine from excess lactose. The galactosylated poloxamine was then freeze dried. A total of 91 mg of the product was recovered. Phenol sulphuric acid assay of the product gave a galactose content of 3.7 mols per TA908 molecule.

### EXAMPLE 3

### Physico Chemical Characterization of Galactosylated Poloxamine 908 and DNA complexes

To a series of scintillation vials containing 1.5 ml Optimem™ and 50 μl plasmid DNA (1 mg/ml) (pCAT—a plasmid containing a CMV promoter and a chloroamphericol acetyltransferase reporter) was added to aliquots of galactosylated poloxamine 908 (10 mg/ml) to give different weight ratios. The complexes were left to stir for 5 mins before determining the particle size using Photon Correlation Spectroscopy (Malvern Instruments).

The complexation of DNA with the galactosylated poloxamine 908 occurs via electrostatic interaction between the phosphate groups of the DNA and the amino group of the copolymer. FIG. 1 shows the changes in size of the complex with increasing ratio of galactosylated poloxamine 908 in the complex.

At lower ratios of poloxamine to DNA, the complexes produced were heterogeneous and with a particle size greater than 500 nm. Increasing the ratio of poloxamine to DNA resulted in the condensation of the DNA, with a decrease in particle size to less than 180 nm. After a ratio of DNA to galactosylated poloxamine of 1:40, no further decrease in particle size was seen.

### EXAMPLE 4

### In Vitro Gene Expression

The human hepatoma cell line HepG2 cells (ECACC no 85011430) was cultured in RPMI medium supplemented with 10% foetal calf serum (FCS) and 1% non essential amino acids and incubated at 37° C., 5% CO₂. The HepG2 cells were seeded onto 12 well tissue culture plates on day 0, using the same culture medium. The cell confluency was about 20%. On day 1, the culture media was removed from the cells and replaced with 1 ml OPTIMEM™ containing 3 μg of the plasmid pCAT complexed with galactosylated poloxamine 908 (gp908). In some of the well plates 100 μl of FCS was also added. Galactosylated poly-L-lysine (gPLL) was used as a comparison. This material does not form part of this invention. It has been described previously by Hashida, et al., J. Control. Rel, 53:301 (1998).

After 5 hours incubation at 37° C., 5% CO₂, the supernatant was removed and replaced with RPMI media containing 1% non essential amino acids and 5% foetal calf serum. After 48 hours, the cells were washed with ice cold phosphate buffered saline (PBS) and lysed using the lysis buffer provided with a CAT ELISA kit (Boehringer Manheim) and the CAT protein measured using CAT ELISA assay (as per the manufacturer's instruction).

The transfection efficiency of the novel gene delivery system was compared with galactosylated poly-L-lysine (gPLL), which has previously been shown to transfect HepG2 cells (Hashida et al., J. Control. Rel., 53:301 (1998)). The transfection efficiency of the complexes was compared in different media, which included foetal calf serum in the transfection media, to assess the protection of the complexes by the block copolymer to prevent degradation of the DNA from serum nucleases. The results of the transfection study are shown in FIG. 2 +L, which compares the transfection efficiency of the different cationic polymers in the HepG2 cell system. For the gp908 system, the presence of serum results surprisingly in a marked increase in transfection compared to the gPLL. The transfection efficiency is doubled with the novel delivery system as compared to gPLL. The transfection efficiency of the gp908 system was only slightly enhanced (about 8%) with the addition of chloroquine encapsulated within the complex. In the absence of the serum, the transfection efficiency of the gp908 system decreased.

The protection of the DNA from degradation by nuclease is believed to be important in achieving efficient gene transfer. The genetic material will be subject to rapid degradation when introduced into the systemic circulation due to serum nuclease activity and capture and subsequent degradation by the cells of the reticulo endothelial system.

The novel non-viral delivery system of the present invention enhances transfection activity in the presence of serum. This may be due to selective adsorption of serum proteins that can provide increased protection as described by Moghimi et al., Biochim. Biophys. Acta, 1179:157 (1993).

In order to achieve cell specificity, the physicochemical properties of the DNA: polymer complexes will be important. For example, it is possible, through formulation, to produce DNA polymer nanoparticles of a size less than 200 nm for liver targeting. This critical size is necessary for the receptor mediated delivery of DNA into the hepatocytes of the liver, because the fenestrations in the liver sinusoid (that provide access to the parenchyma) are of a size of less than about 250 nm.

Once inside the cell, the localisation of the complex, its resistance to cellular nucleases and the degree to which the complexed genetic material is expressed combine to determine the overall efficiency of the gene transfer. The presence of chloroquine only increased the transfection efficiency of the delivery system by 8%. Consequently, the system can be termed chloroquine independent in its effect.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition comprising:
(a) a nucleic acid or an oligonucleotide; and
(b) an aminated polyalkylene block copolymer composed of polyoxyethylene and polyoxypropylene blocks in which only terminal hydroxy groups have been substituted by an amino group to provide the block with a positive charge.

2. A composition according to claim 1, wherein the copolymer carries a targeting moiety.

3. A composition according to claim 2, wherein the targeting moiety is a sugar.

4. A composition according to claim 3, wherein the sugar is galactose.

5. A composition according to claim 1, comprising nanoparticles comprising the copolymer and a nucleic acid or an oligonucleotide and having a particle size of 500 nm or less.

6. A composition according to claim 1, wherein the ratio of nucleic acid or oligonucleotide to polymer is from 1:5000 to 1:5 on a weight ratio basis.

7. A method for the delivery of a nucleic acid or an oligonucleotide to cells which comprises administering a composition as defined in claim 1.

8. A glycosylated block copolymer.

9. A copolymer according to claim 8, comprising a hydrophilic block and a hydrophobic block.

10. A copolymer according to claim 8, comprising a polyoxyethylene block and a polyoxypropylene block.

11. A method for the delivery of a nucleic acid or an oligonucleotide to a cell, comprising forming a medicament with a glycosylated block copolymer according to claim 9.

12. A method of targetting a nucleic acid or an oligonucleotide to the liver, comprising combining the nucleic acid or oligonucleotide with a glycosylated block copolymer according to claim 9.
